(19) United States
(12) Patent Application Publication
(10) Pub. No.: US 2020/0171025 A1
(43) Pub. Date: Jun. 4, 2020

(54) PHARMACEUTICAL COMPOSITION COMPRISING BREXPIPRAZOLE AND PROCESS FOR PREPARATION THEREOF

(71) Applicants: Srinivasa Rao Parella, Hyderabad (IN); Nilesh Bhandari, Hyderabad (IN); Nagaprasad Vishnubhotla, Hyderabad (IN); Sivakumaran Meenakshisunderam, Hyderabad (IN)

(72) Inventors: Srinivasa Rao Parella, Hyderabad (IN); Nilesh Bhandari, Hyderabad (IN); Nagaprasad Vishnubhotla, Hyderabad (IN); Sivakumaran Meenakshisunderam, Hyderabad (IN)

(21) Appl. No.: 16/698,617

(22) Filed: Nov. 27, 2019

(30) Foreign Application Priority Data
Nov. 29, 2018 (IN) 201841045085

(51) Int. Cl. A61K 31/497; A61K 9/20

(52) U.S. Cl. CPC A61K 31/497; A61K 9/2013; A61K 9/2059; A61K 9/2054

(57) ABSTRACT

The present invention relates to a pharmaceutical composition comprising brexpiprazole or a pharmaceutically acceptable salt thereof and at least one pharmaceutically acceptable excipient, wherein particle size of brexpiprazole or pharmaceutically acceptable salt thereof is D₉₀ from about 10 μm to about 40 μm, particularly D₉₀ is in the range from about 15 μm to about 30 μm and process of preparation of said pharmaceutical composition and its use for the adjunctive treatment of major depressive disorder (MDD) and treatment of schizophrenia.

### CROSS REFERENCE TO RELATED APPLICATIONS

This application claims priority from an Indian Patent Application IN 201841045085 filed on Nov. 29, 2018

### FIELD OF THE INVENTION

The present invention relates to a solid pharmaceutical composition comprising brexpiprazole or a pharmaceutically acceptable salt thereof and at least one pharmaceutically acceptable excipient and its preparation process and method of using the same.

### BACKGROUND OF THE INVENTION

Antipsychotic drugs in general are known as major tranquilisers and neuroleptics and are used to treat psychiatric conditions. Brexpiprazole is an atypical antipsychotic drug which acts as a dopamine D2 receptor partial agonist as well as a serotonin reuptake inhibitor. The chemical name of brexpiprazole is 7-{4-[4-(1-Benzothiophen-4-yl)piperazin-1-yl]butoxy}quinolin-2(1H)-one of the formula C₂₅H₂₇N₃O₂S, molecular weight of 433.57 and having the following chemical structure:

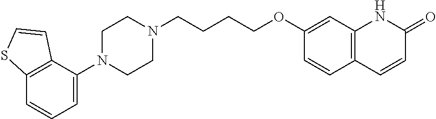 [SMILES: O=c1ccc2ccc(OCCCCN3CCN(c4cccc5sccc45)CC3)cc2[nH]1]

Brexpiprazole tablet formulation was first approved in the USA on Jul. 10, 2015 under the brand name REXULTI® for Otsuka Pharma Co. Ltd. It is available in 0.25 mg, 0.5 mg, 1 mg, 2 mg, 3 mg and 4 mg strengths and mainly used in the adjunctive treatment of major depressive disorder (MDD) and treatment of schizophrenia.

As per Biopharmaceutics classification system, Brexpiprazole is a class II drug and has low aqueous solubility and high permeability compound and poses technical challenges in the formulation development. it is unstable under radiant (i.e. light) energy.

Following patent/patent publications pertain to various formulations of Brexpiprazole:

U.S. Pat. Nos. 7,888,362, 8,349,840, 8,618,109 describes Brexpiprazole product as well as its use in treatment of major depressive disorder (MDD) and schizophrenia.

U.S. pat. No. 10,30,7419 and its family equivalents U.S. publication No's 20140234417, 20160158227, 20170165258 & US20190240215 discloses brexpiprazole tablet composition and method for producing a tablet. The tablet composition disclosed in said U.S. applications comprises an excipients comprising a diluents (lactose, corn starch, and microcrystalline cellulose), a binder (hydroxypropyl cellulose), a disintegrant (low-substituted hydroxypropyl cellulose, croscarmellose sodium, and sodium carboxymethyl starch), and a lubricant (magnesium stearate) and also discloses granulation (Dry & Wet) methods for preparation of brexpiprazole tablet and wherein said tablet composition exhibits excellent disintegration, storage stability and higher photostability. The said U.S. patent and patent applications emphasized on use of i) Hydroxypropyl cellulose as a preferred binder and ii) Use of coating layer to attain said properties.

Indian patent application No. 201611038602 discloses solid oral pharmaceutical composition of comprising brexpiprazole substantially free of disintegrants and/or binders.

Indian patent application No. 201721002429 discloses pharmaceutical composition comprising brexpiprazole, a binder and a stabilizer, wherein the binder is selected from povidone or crospovidone and stabilizer selected from EDTA salts and ascorbic acid or its derivatives.

The prior art compositions are useful in minimizing the problems associated with brexpiprazole composition but there is still need to design pharmaceutical composition of brexpiprazole tablet that is economical and commercially viable and having excellent dissolution, storage stability and high photostability.

The inventors of present invention surprisingly found that use of hydroxypropyl methylcellulose (HPMC) as a binder in a pharmaceutical composition of brexpiprazole or pharmaceutically acceptable salt thereof exhibits excellent dissolution and storage stability and it is comparable to marketed reference product REXULTI®.

Further the inventors of present invention surprisingly found that a pharmaceutical composition of brexpiprazole or pharmaceutically acceptable salt thereof having brexpiprazole particles D₉₀ from about 10 μm to about 40 μm provides improved dissolution and bioavailability of brexpiprazole when compared to marketed reference product REXULTI®.

### SUMMARY OF THE INVENTION

In one aspect, there is provided a pharmaceutical composition comprising brexpiprazole or a pharmaceutically acceptable salt thereof and at least one pharmaceutically acceptable excipient.

In another aspect, there is provided a pharmaceutical composition comprising brexpiprazole or pharmaceutically acceptable salt thereof and at least one pharmaceutically acceptable excipients including diluent, binder, disintegrant, lubricant, wherein hydroxypropyl methylcellulose (HPMC) is used as a binder.

In another aspect, an invention provides a solid pharmaceutical composition comprising:
a) 0.1-10% w/w of brexpiprazole or its pharmaceutically acceptable salt thereof, wherein brexpiprazole has a particle size distribution such that more than 90% of the particles are between 10 μm to 40 μm;
b) 70-95% w/w of diluent selected from lactose monohydrate, corn starch and microcrystalline cellulose or combination thereof;
c) 5-15% w/w of croscarmellose sodium as disintegrant;
d) 0.5-5% w/w of hydroxypropyl methyl cellulose as binder;
e) 0.1-2% w/w of magnesium stearate as lubricant;
Wherein at least 90% of brexpiprazole dissolves within 30 minutes in a 900 ml of pH 4.3 acetate buffer at a temperature of 37±0.5° C. using a USP apparatus-2 at a paddle rotation of about 50 rpm.

In another aspect, an invention provides a solid pharmaceutical composition comprising:
a) 0.1-10% w/w of brexpiprazole or its pharmaceutically acceptable salt thereof, wherein brexpiprazole has a particle size distribution such that more than 90% of the particles are between 10 μm to 40 μm;
b) 70-95% w/w of lactose monohydrate, corn starch and microcrystalline cellulose or combination thereof,
c) 5-15% w/w of croscarmellose sodium;
d) 0.5-5% w/w of hydroxypropyl methyl cellulose;
e) 0.1-2% w/w of magnesium stearate;
Wherein weight ratio of lactose monohydrate to corn starch to microcrystalline cellulose is in the range of 4:2:1 to 5:2:1 and the composition does not contains any detectable impurities by weight relative to brexpiprazole when measured by HPLC method after storage for 6 months at 40° C./75% relative humidity.

In another aspect, there is provided a solid pharmaceutical composition comprising:
a) 0.25-4 mg of brexpiprazole or its pharmaceutically acceptable salt thereof, wherein brexpiprazole or a salt thereof has a particle size distribution such that more than 90% of the particles are between 10 μm to 40 μm;
b) 65-85 mg of diluent selected from lactose, corn starch and microcrystalline cellulose and combination thereof;
c) 5-14 mg of croscarmellose sodium;
d) 0.5-5 mg of hydroxypropyl methyl cellulose;
e) 0.1-2 mg of magnesium stearate;
Wherein the weight ratio of croscarmellose sodium to brexpiprazole is 1:0.020 to 1:0.5 and at least 90% of brexpiprazole dissolves within 30 minutes in a 900 ml of pH 4.3 acetate buffer at a temperature of 37±0.5° C. using a USP apparatus-2 at a paddle rotation of about 50 rpm.

In another aspect, there is provided a solid pharmaceutical composition comprising:
a) 0.25-4 mg of brexpiprazole;
b) 40-50 mg of lactose monohydrate;
c) 20 mg of corn starch;
d) 10 mg of microcrystalline cellulose;
e) 10 mg of croscarmellose sodium;
f) 2 mg of hydroxypropyl methyl cellulose;
g) 0.6 mg of magnesium stearate;
Wherein brexpiprazole has a particle size distribution such that more than 90% of the particles are between 15 μm to 25 μm and the weight ratio of hydroxypropyl methyl cellulose to brexpiprazole is in the range of 1:0.125 to 1:2 and at least 90% of brexpiprazole dissolves within 30 minutes in a 900 ml of pH 4.3 acetate buffer at a temperature of 37±0.5° C. using a USP apparatus-2 at a paddle rotation of about 50 rpm.

In another aspect, there is provided a solid pharmaceutical composition comprising:
a) 0.2-5% w/w of brexpiprazole having a particle size distribution such that more than 90% of the particles are between 10 μm to 40 μm;
b) 43-57% w/w of lactose monohydrate;
c) 20-23% w/w of corn starch;
d) 10-12% w/w of microcrystalline cellulose;
e) 10-12% w/w of croscarmellose sodium;
f) 2-2.5% w/w/of hydroxypropyl methyl cellulose;
g) 0.63-0.67% w/w of magnesium stearate;
Wherein said solid pharmaceutical composition is a tablet or capsule, if the solid pharmaceutical composition is a tablet, then it is coated with film coating material, the weight ratio of film coating material to brexpiprazole is 1:0.01 to 1:2.

In another aspect, an invention provides a solid pharmaceutical composition comprising:
a) 0.25-4 mg of brexpiprazole or its pharmaceutically acceptable salt thereof, wherein brexpiprazole or a salt thereof has a particle size distribution such that more than 90% of the particles are between 10 μm to 40 μm;
b) 65-85 mg of diluent selected from lactose, corn starch and microcrystalline cellulose and combination thereof;
c) 5-14 mg of croscarmellose sodium;
d) 0.5-5 mg of hydroxypropyl methyl cellulose;
e) 0.1-2 mg of magnesium stearate;
Wherein said composition is compressed to get a tablet and coated with a 3-5% w/w of a film coating material and the weight ratio of lactose monohydrate to film coating material is about 1:0.01 to 1:0.1.

In another aspect, there is provided a solid pharmaceutical composition comprising:
a) 0.25-4 mg of brexpiprazole;
b) 40-50 mg of lactose monohydrate as diluent;
c) 20 mg of corn starch as diluent;
d) 10 mg of microcrystalline cellulose as diluent;
e) 10 mg of croscarmellose sodium as disintegrant;
f) 2 mg of hydroxypropyl methyl cellulose as binder;
g) 0.6 mg of magnesium stearate as lubricant;
wherein said composition is compressed to get a tablet dosage form and coating the tablet with film coating material comprising hydroxypropyl methyl cellulose, titanium dioxide, talc ferrosoferric oxide, iron oxide yellow, iron oxide red, FD&C blue, red and yellow colors.

In another aspect, an invention provides the process for producing a pharmaceutical solid dosage form which comprises steps of:
a) mixing brexpiprazole with excipients selected from lactose, corn starch, microcrystalline cellulose and croscarmellose sodium;
b) granulating the mixture obtained in step “a” using binder solution comprising hydroxypropyl methyl cellulose;
c) drying the granules obtained in step “b” to get a LOD % not more than 9.0% w/w at 105° C.;
d) optionally milling and then blending the granules with magnesium stearate, which is further compressed to get tablet dosage form and it is optionally film coated.

In another aspect, there is provided a method of using a pharmaceutical composition of comprising brexpiprazole or pharmaceutically acceptable salt thereof for the treatment of central nervous system diseases. In particular the pharmaceutical composition is used in the adjunctive treatment of major depressive disorder (MDD) and treatment of schizophrenia.

### DETAILED DESCRIPTION OF THE INVENTION

The term “composition”, as in solid pharmaceutical composition, is intended to encompass a drug product comprising Brexpiprazole or its pharmaceutically acceptable salts, esters, solvates, polymorphs, enantiomers or mixtures thereof, and other inert ingredient(s) (pharmaceutically acceptable excipients). Such pharmaceutical compositions are synonymous with “formulation” and “dosage form”. Pharmaceutical composition of the invention include, but is not limited to, granules, tablets, immediate release tablets, caplets, capsules (immediate or modified release) (hard and soft or liquid filled soft gelatin capsules) and the like. Preferably, the pharmaceutical composition refers to tablets and capsules. More preferably, the pharmaceutical composition refers to immediate release oral tablets, which may be uncoated or film coated.

The term “excipient”, means a pharmacologically inactive component such as a diluent, binder, disintegrant, lubricant, coloring agent or the like. The excipients that are useful in preparing a pharmaceutical composition are generally safe, non-toxic and are acceptable for veterinary as well as human pharmaceutical use. Reference to an excipient includes both one and more than one such excipient.

The term “Brexpiprazole” is used in broad sense to include not only “Brexpiprazole” per se but also its pharmaceutically acceptable salts, solvates, hydrates, enantiomers, derivatives, isomers, polymorphs, prodrugs thereof, and also its various crystalline and amorphous forms.

The amount of Brexpiprazole according to the invention may be present at 0.1 to 10% by weight based on total weight of the composition, preferably, 0.1% to 8% w/w, more preferably 0.2% to 6%, most preferably 0.25% to 5% w/w or 0.26% to 4.32% w/w.

The term “pharmaceutically acceptable salt” refers to salts derived from a variety of organic and inorganic counter ions including fumarate, maleate, phosphate, L-tartrate, citrate, acetate, oxalate, and sulfate.

The term “% by weight of the tablet” refers to the percentage by weight of each ingredient in the core tablet, including any exterior coatings.

The term “loss on drying” is a widely used test method to determine the water content of a sample, although occasionally it may refer to the loss of any volatile matter from the sample. The step of drying is obtained by heating the granules to a temperature above room temperature and maintaining the elevated temperature, until the LOD of the granules reaches a desired value. The granules are typically characterized by having a solvent loss on drying at 105° C. of less than 9% w/w, preferably less than 8% w/w, 7.5% w/w, 7.4% w/w, 7.3% w/w, 7.2% w/w, 7.1% w/w and 7% w/w.

The term “impurity” refers to undesired contents present or produced in a pharmaceutical composition.

The dissolution is performed as per conditions mentioned or provided in office of generic drugs dissolution database and as determined by the USP. The dissolution profile of tablets dosage form was measured in 900 ml of acetate buffer, pH 4.3 using a USP II apparatus (Paddle) at a temperature of 37±0.5° C. and a rotation speed of 50 revolutions per minute.

The term “treating” or “treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder or delaying, inhibiting or decreasing the likelihood of the progression of the disease, disorder or syndrome.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent.

The term “particle(s)” as used herein refers to individual particles of brexpiprazole or pharmaceutically acceptable salt thereof, whether the particles exist singly or are agglomerated. The term “particle size” of brexpiprazole having a particle size distribution such that more than 90% of the particles are between 10 μm to 40 μm, preferably 15 μm to 30 μm, more preferably 16 μm to 25 μm and most preferably 18 μm to 22 μm or 19 μm or 20 μm or 21 μm. The particle size of brexpiprazole was measured using a Malvern light scattering technique.

The term “stable” as used herein refers to formulations that substantially retain the label amount of the therapeutically active ingredient during storage for commercially relevant times, and the drug-related impurity contents in the formulations remain within the acceptable limit.

The use of the terms “a” and “an” and “the” and similar referents in the context of describing the invention (especially in the context of the following claims) are to be construed to cover both the singular and the plural, unless otherwise indicated herein or clearly contradicted by context.

Throughout this specification and the appended claims, it is to be understood that the words “comprise”, “have”, “contain” and “include” and variations such as “comprises”, “comprising”, “having”, “containing” “includes”, “including” are to be interpreted inclusively, unless the context requires otherwise. That is, the use of these words may imply the inclusion of an element or elements not specifically recited.

The term “diluent” or “filler” as used herein is defined as an inert agent designed to increase the weight and/or the size of the pharmaceutical composition, for example in the case of a tablet. Examples of diluents according to present invention include, but not limited to group comprising of microcrystalline cellulose, carboxymethyl cellulose (carmellose), sodium carboxymethyl cellulose (carmellose sodium), corn starch, potato starch, anhydrous lactose, lactose monohydrate and the mixtures thereof. Preferably the diluents are selected from lactose monohydrate, corn starch, microcrystalline cellulose or combination thereof. The diluents according to present invention may be present in an amount from about 10% to about 95% by weight with respect to total weight of the pharmaceutical composition, preferably 70% to 90% w/w, most preferably 75% to 85% w/w.

The combination of diluents are used which are selected from lactose monohydrate, corn starch and microcrystalline cellulose or combination thereof. The weight ratio of lactose monohydrate to corn starch is about 2:1 to 2.5:1, preferably 2.15:1 to 2.4:1. The weight ratio of corn starch to microcrystalline cellulose is 1.5:0.5 to 2.5:1.5, preferably 1.8:0.8 to 2.2:1.2, most preferably 1.9:0.9 or 2:1 or 2.1:1.1. The weight ratio of lactose monohydrate to microcrystalline cellulose is 4:1 to 5:1, preferably 4.2:1 to 4.8:1, most preferably 4.3:1 to 4.7:1. The weight ratio of lactose monohydrate to corn starch to microcrystalline cellulose is 4:2:1 to 5:2:1.

The weight ratio of brexpiprazole to lactose monohydrate is 0.006:1 to 0.09:1, preferably 0.05:1 to 0.08:1. The weight ratio of brexpiprazole to corn starch is 0.01:1 to 0.4:1, preferably 0.012:1 to 0.2:1. The weight ratio of microcrystalline cellulose to brexpiprazole is 1:0.020 to 1:0.5, preferably 1:0.025 to 1:0.4. The weight ratio of brexpiprazole to lactose monohydrate to corn starch to microcrystalline cellulose is 0.006:1:0.5:0.25 to 0.08:1:0.4:0.2.

The term “binder” as used herein is defined as an agent able to bind particles which cannot be bound only by a compression force. The binder may be present in the form of a single compound or in the form of a mixture of compounds. Examples of Binders according to present invention include, but not limited to group comprising of pregelatinized starch; partially pregelatinized starch; cellulose or a derivative thereof such as microcrystalline cellulose, methyl cellulose, ethyl cellulose, sodium carboxymethyl cellulose (carmellose sodium), hydroxyethyl cellulose, hydroxyethyl methyl cellulose, hydroxypropyl methyl cellulose (hypromelloses such as hypromellose 2208, hypromellose 2906, and hypromellose 2910) and mixture thereof. Preferably the binder is hydroxypropyl methylcellulose (HPMC). The binder according to present invention may be present in an amount from about 0.5% to about 5.0% by weight with respect to total weight of the pharmaceutical composition, preferably, 1% to 4% or 2% or 2.1% or 2.2% or 2.5% or 3% w/w.

The weight ratio of hydroxypropyl methyl cellulose to brexpiprazole is in the range of 1:0.125 to 1:2.

The term “disintegrant” as used herein is defined as an accelerating agent of the disintegration of the tablet and the dispersion of the active ingredient in water or gastrointestinal fluids. Examples of disintegrants according to present include, but not limited to group comprising of starch or a derivative thereof such as sodium carboxymethyl starch (sodium starch glycolate); cellulose or a derivative thereof such as microcrystalline cellulose, carboxymethyl cellulose (carmellose), calcium carboxymethyl cellulose (carmellose calcium), croscarmellose sodium and mixture thereof. Preferably the disintegrant is croscarmellose sodium. The disintegrant according to present invention may be present in an amount from about 1% to about 15% by weight with respect to total weight of the pharmaceutical composition, preferably 5% to 15% w/w, more preferably 8 to 13% w/w or 9% or 10% or 10.5% or 10.8% or 11% w/w.

The weight ratio of croscarmellose sodium to brexpiprazole is 1:0.020 to 1:0.5, preferably 1:0.025 to 1:0.4. The weight ratio of croscarmellose sodium to hydroxypropyl methyl cellulose is 1:0.1 to 1:0.4, preferably 1:0.15 to 1:0.3, most preferably 1:0.2.

The term “lubricant” as used herein is defined as an agent able to decrease adhesion of a powder to punches and friction between particles. The lubricant may be present in the pharmaceutical composition in the form of a single compound or in the form of a mixture of compounds. Examples of lubricants according to present invention include, but not limited to group comprising of stearic acid, aluminum stearate, calcium stearate, and magnesium stearate; carnauba wax; glycerol ester of fatty acid and mixture thereof. Preferably the lubricant is magnesium stearate.

The lubricant according to present invention may be present in an amount from about 0.1% to about 10% by weight with respect to total weight of the pharmaceutical composition, preferably 0.1 to 2% w/w, more preferably 0.4 to 0.8% w/w or 0.5% or 0.6% or 0.65% or 0.66% or 0.7% w/w.

The weight ratio of magnesium stearate to brexpiprazole is 1:0.2 to 1:7, preferably 1:0.4 to 1:67.

The term “coated tablet” as used herein is defined as an tablet provided with a coating layer is preferable to achieve long-term storage stability and prevent degradation due to light and the like. The coating layer comprise pharmaceutical additives, such as a coating agent, plasticizer, dispersant, defoaming agent, and the like, usually used for coating (for providing a coat to) orally administrable pharmaceutical preparations. Colorants are added to the coating agent for coating the tablet. Examples of colorants include but are not limited to iron oxides such as red ferric oxide, yellow ferric oxide, and black iron oxide; titanium oxide; beta-carotene; food blue No. 2; food blue No. 2 aluminium lake; and riboflavin and the like. Among these, an iron oxide is more preferable. Examples of film coating material includes Opadry which is Colorcon's customized, one-step film coating system which combines polymer, plasticizer, opacifier and pigment, as required, in a dry concentrate. Examples of Opadry coating material is selected from but not limited to Opadry grey, Opadry pink, Opadry red, Opadry blue, Opadry tan, Opadry Maroon, Opadry orange, Opadry brown, Opadry purple, Opadry white, Opadry yellow, Opadry green and the like, preferably Opadry coating material used according to the present invention is Opadry grey, Opadry pink, Opadry tan, Opadry blue, Opadry red and Opadry maroon.

Composition of Opadry grey contains hydroxypropyl methyl cellulose, titanium dioxide, talc, ferrosoferric oxide and iron oxide yellow; Opadry pink contains hydroxypropyl methyl cellulose, titanium dioxide, talc, iron oxide yellow and iron oxide red; Opadry tan contains hydroxypropyl methyl cellulose, titanium dioxide, talc, ferrosoferric oxide, iron oxide yellow and iron oxide red; Opadry blue contains hydroxypropyl methyl cellulose, titanium dioxide, talc, FD&C blue, indigo caramine aluminium lake; Opadry red contains hydroxypropyl methyl cellulose, titanium dioxide, talc, FD&C yellow, FD&C red; Opadry maroon contains hydroxypropyl methyl cellulose, titanium dioxide, talc, FD&C red, FD&C blue and indigo caramine aluminium lake.

The weight ratio of film coating material to brexpiprazole is 1:0.01 to 1:2, preferably 1:0.05 to 1:1.5, more preferably 1:0.07 to 1:1.4 and the weight ratio of film coating material to hydroxypropyl methyl cellulose as binder is 1:0.50 to 1:0.60, preferably 1:0.53 to 1:0.58 or 1:0.54 or 1:0.55 or 1:0.56 or 1:0.57. The weight ratio of lactose monohydrate to film coating material is about 1:0.01 to 1:0.1, preferably 1:0.03 to 1:0.1 or 1:0.04 to 1:0.09 or 1:0.07 to 1:0.08. The weight ratio of hydroxypropyl methyl cellulose to brexpiprazole to film coating material composition is 1:0.12:1.8 to 1:2:1.8.

In one embodiment, the invention relates to a solid pharmaceutical composition comprising brexpiprazole or its pharmaceutically acceptable salts, esters, solvates, polymorphs, enantiomers or mixtures thereof with one or more pharmaceutically acceptable excipient.

In another embodiment, the invention relates to a solid pharmaceutical composition comprising brexpiprazole or pharmaceutically acceptable salt thereof and at least one pharmaceutically acceptable excipients including diluent, binder, disintegrant, lubricant, wherein hydroxypropyl methylcellulose (HPMC) is used as a binder.

In another embodiment, an invention provides a solid pharmaceutical composition comprising:
a) 0.1-10% w/w of brexpiprazole or its pharmaceutically acceptable salt thereof, wherein brexpiprazole has a particle size distribution such that more than 90% of the particles are between 10 μm to 40 μm;
b) 70-95% w/w of diluent selected from lactose monohydrate, corn starch and microcrystalline cellulose or combination thereof;
c) 5-15% w/w of croscarmellose sodium as disintegrant;
d) 0.5-5% w/w of hydroxypropyl methyl cellulose as binder;
e) 0.1-2% w/w of magnesium stearate as lubricant;
Wherein at least 90% of brexpiprazole dissolves within 30 minutes in a 900 ml of pH 4.3 acetate buffer at a temperature of 37±0.5° C. using a USP apparatus-2 at a paddle rotation of about 50 rpm.

In another embodiment, an invention provides a solid pharmaceutical composition comprising:
a) 0.1-10% w/w of brexpiprazole or its pharmaceutically acceptable salt thereof, wherein brexpiprazole has a particle size distribution such that more than 90% of the particles are between 10 μm to 40 μm;
b) 70-95% w/w of lactose monohydrate, corn starch and microcrystalline cellulose or combination thereof,
c) 5-15% w/w of croscarmellose sodium;
d) 0.5-5% w/w of hydroxypropyl methyl cellulose;
e) 0.1-2% w/w of magnesium stearate;
Wherein weight ratio of lactose monohydrate to corn starch to microcrystalline cellulose is in the range of 4:2:1 to 5:2:1 and the composition does not contains any detectable impurities by weight relative to brexpiprazole when measured by HPLC method after storage for 6 months at 40° C./75% relative humidity.

In another embodiment, an invention provides a solid pharmaceutical composition comprising:
a) 0.25-4 mg of brexpiprazole or its pharmaceutically acceptable salt thereof, wherein brexpiprazole or a salt thereof has a particle size distribution such that more than 90% of the particles are between 10 μm to 40 μm;
b) 65-85 mg of diluent selected from lactose, corn starch and microcrystalline cellulose and combination thereof;
c) 5-14 mg of croscarmellose sodium;
d) 0.5-5 mg of hydroxypropyl methyl cellulose;
e) 0.1-2 mg of magnesium stearate;
Wherein at least 90% of brexpiprazole dissolves within 30 minutes in a 900 ml of pH 4.3 acetate buffer at a temperature of 37±0.5° C. using a USP apparatus-2 at a paddle rotation of about 50 rpm.

In another embodiment, there is provided a solid pharmaceutical composition comprising:
a) 0.25-4 mg of brexpiprazole or its pharmaceutically acceptable salt thereof, wherein brexpiprazole or a salt thereof has a particle size distribution such that more than 90% of the particles are between 10 μm to 40 μm;
b) 65-85 mg of diluent selected from lactose, corn starch and microcrystalline cellulose and combination thereof;
c) 5-14 mg of croscarmellose sodium;
d) 0.5-5 mg of hydroxypropyl methyl cellulose;
e) 0.1-2 mg of magnesium stearate;
Wherein the weight ratio of croscarmellose sodium to brexpiprazole is 1:0.020 to 1:0.5 and at least 90% of brexpiprazole dissolves within 30 minutes in a 900 ml of pH 4.3 acetate buffer at a temperature of 37±0.5° C. using a USP apparatus-2 at a paddle rotation of about 50 rpm.

In another embodiment, there is provided a solid pharmaceutical composition comprising:
a) 0.25-4 mg of brexpiprazole;
b) 40-50 mg of lactose monohydrate;
c) 20 mg of corn starch;
d) 10 mg of microcrystalline cellulose;
e) 10 mg of croscarmellose sodium;
f) 2 mg of hydroxypropyl methyl cellulose;
g) 0.6 mg of magnesium stearate;
Wherein brexpiprazole has a particle size distribution such that more than 90% of the particles are between 15 μm to 25 μm and the weight ratio of hydroxypropyl methyl cellulose to brexpiprazole is in the range of 1:0.125 to 1:2 and at least 90% of brexpiprazole dissolves within 30 minutes in a 900 ml of pH 4.3 acetate buffer at a temperature of 37±0.5° C. using a USP apparatus-2 at a paddle rotation of about 50 rpm.

In another embodiment, there is provided a solid pharmaceutical composition comprising:
a) 0.2-5% w/w of brexpiprazole having a particle size distribution such that more than 90% of the particles are between 10 μm to 40 μm;
b) 43-57% w/w of lactose monohydrate;
c) 20-23% w/w of corn starch;
d) 10-12% w/w of microcrystalline cellulose;
e) 10-12% w/w of croscarmellose sodium;
f) 2-2.5% w/w/ of hydroxypropyl methyl cellulose;
g) 0.63-0.67% w/w of magnesium stearate;
Wherein said solid pharmaceutical composition is a tablet or capsule, if the solid pharmaceutical composition is a tablet, then it is coated with film, coating material, the weight ratio of film coating material to brexpiprazole is 1:0.01 to 1:2.

In another embodiment, an invention provides a solid pharmaceutical composition comprising:
a) 0.25-4 mg of brexpiprazole or its pharmaceutically acceptable salt thereof, wherein brexpiprazole or a salt thereof has a particle size distribution such that more than 90% of the particles are between 10 μm to 40 μm;
b) 65-85 mg of diluent selected from lactose, corn starch and microcrystalline cellulose and combination thereof;
c) 5-14 mg of croscarmellose sodium;
d) 0.5-5 mg of hydroxypropyl methyl cellulose;
e) 0.1-2 mg of magnesium stearate;
Wherein said composition is compressed to get a tablet and coated with a 3-5% w/w of a film coating material and the weight ratio of lactose monohydrate to film coating material is about 1:0.01 to 1:0.1.

In another embodiment, there is provided a solid pharmaceutical composition comprising:
a) 0.25-4 mg of brexpiprazole;
b) 40-50 mg of lactose monohydrate as diluent;
c) 20 mg of corn starch as diluent;
d) 10 mg of microcrystalline cellulose as diluent;
e) 10 mg of croscarmellose sodium as disintegrant;
f) 2 mg of hydroxypropyl methyl cellulose as binder;
g) 0.6 mg of magnesium stearate as lubricant;
wherein said composition is compressed to get a tablet dosage form and coating the tablet with film coating material comprising hydroxypropyl methyl cellulose, titanium dioxide, talc ferrosoferric oxide, iron oxide yellow, iron oxide red, FD&C blue, red and yellow colors.

In another embodiment, an invention provides the process for producing a pharmaceutical solid dosage form which comprises steps of:
a) mixing brexpiprazole with excipients selected from lactose, corn starch, microcrystalline cellulose and croscarmellose sodium;
b) granulating the mixture obtained in step “a” using binder solution comprising hydroxypropyl methyl cellulose;
c) drying the granules obtained in step “b” to get a LOD % not more than 9.0% w/w at 105° C. and optionally milling and then blending the granules with magnesium stearate, which is further compressed to get tablet dosage form and it is optionally film coated.

In another embodiment, there is provided a method of using a pharmaceutical composition of comprising brexpiprazole or pharmaceutically acceptable salt thereof for the treatment of central nervous system diseases. In particular the pharmaceutical composition is used in the adjunctive treatment of major depressive disorder (MDD) and treatment of schizophrenia.

Surprisingly, it has been found that the pharmaceutical composition of the present invention has been found to have improved stability and dissolution profile coupled with simple manufacturing process at industrial scale and it is bioequivalence to commercially available counterpart tablets REXULTI®.

The following examples serve to illustrate the embodiments of the present invention. However, they do not intend to limit the scope of the invention. It is obvious to those skilled in the art to find out the composition for other dosage forms and substitute the equivalent excipients as described in this specification or with the one known to the industry.

### Example 1-3 Represents Brexpiprazole Tablets According to the Present Invention

### Example 1

| | | | | |
|---|---|---|---|---|
| | | 1A | 1B | 1C |
| No | Ingredients | Mg/Tab | Mg/Tab | Mg/Tab |

Core Tablet; Intragranular

| | | | | |
|---|---|---|---|---|
| 1 | Brexpiprazole | 4.00 | 4.00 | 4.00 |
| 2 | Lactose Monohydrate | 50.00 | 42.40 | 44.00 |
| 3 | Corn Starch | 18.00 | 21.00 | 20.00 |
| 4 | Microcrystalline Cellulose | 11.50 | 10.00 | 09.40 |
| 5 | Croscarmellose sodium/ | 5.00 | 10.00 | 10.00 |
| | Sodium carboxymethyl starch | | | |

Binder Solution

| | | | | |
|---|---|---|---|---|
| 6 | Hydroxypropyl methylcellulose | 1.00 | 2.00 | 2.00 |
| 7 | Purified Water | Q.S. | Q.S. | Q.S. |

Extragranular

| | | | | |
|---|---|---|---|---|
| 8 | Magnesium Stearate | 0.50 | 0.60 | 0.60 |
| | Core tablet weight | 90.00 | 90.00 | 90.00 |

Film coating

| | | | | |
|---|---|---|---|---|
| 9 | Opadry Maroon* | 2.70 | 2.70 | 2.70 |
| 10 | Purified Water | Q.S. | Q.S. | Q.S. |
| | Total tablet weight | 92.70 | 92.70 | 92.70 |

*Opadry Maroon contains HPMC, Titanium dioxide, Talc, FD & C red, FD & C blue.

### Manufacturing Process:

1. Brexpiprazole, corn starch, croscarmellose sodium/sodium carboxymethyl starch (Sodium starch glycolate), microcrystalline cellulose were co-sifted through suitable sieve to form a blend and mix well;
2. Binder solution was prepared by dissolving hydroxypropyl methylcellulose (HPMC) in purified water;
3. Granulation: granulation of blend obtained in step (1) was done by using binder solution of obtained in step (2);
4. Granules formed in step (3) were dried at suitable temperature to obtain suitable loss on drying (LOD);
5. Dried granules of step (4) were sifted and milled and passed through suitable sieve;
6. Extra granular ingredient magnesium stearate was sifted through suitable sieve;
7. Granules of step (5) and magnesium stearate of step (6) were mixed and blended in a suitable blender;
8. Blend of step (7) was compressed using suitable tooling to form uncoated tablets and were film coated.

### Example 2

| | | | | |
|---|---|---|---|---|
| | | 2A | 2B | 2C |
| No | Ingredients | % w/w | % w/w | % w/w |

Core Tablet; Intragranular

| | | | | |
|---|---|---|---|---|
| 1 | Brexpiprazole | 4.31 | 4.31 | 4.31 |
| 2 | Lactose Monohydrate | 54.0 | 45.73 | 47.48 |
| 3 | Corn Starch | 19.4 | 22.65 | 21.58 |
| 4 | Microcrystalline Cellulose | 12.4 | 10.8 | 10.14 |
| 5 | Croscarmellose sodium/ | 5.4 | 10.8 | 10.79 |
| | Sodium carboxymethyl starch | | | |

Binder Solution

| | | | | |
|---|---|---|---|---|
| 6 | Hydroxypropyl methylcellulose | 1.07 | 2.16 | 2.15 |
| 7 | Purified Water | Q.S. | Q.S. | Q.S. |

Extragranular

| | | | | |
|---|---|---|---|---|
| 8 | Magnesium Stearate | 0.52 | 0.65 | 0.65 |

Film coating

| | | | | |
|---|---|---|---|---|
| 9 | Opadry Maroon* | 2.9 | 2.9 | 2.9 |
| 10 | Purified Water | Q.S. | Q.S. | Q.S. |

*Opadry Maroon contains HPMC, Titanium dioxide, Talc, FD & C red, FD & C blue.

### Manufacturing Process:

Manufacturing process is similar to example 1.

### Example 3

| | | Quantity per unit | |
|---|---|---|---|
| No | Ingredients | (mg) | (% w/w) |

Intra-granular

| | | | | | | | | |
|---|---|---|---|---|---|---|---|---|
| 1 | Brexpiprazole | 0.25 | 0.50 | 1.00 | 2.00 | 3.00 | 4.00 | 80-90 |
| 2 | Lactose monohydrate | 47.15 | 51.59 | 41.76 | 49.94 | 39.96 | 43.40 | 0.1-1 |
| 3 | Corn starch | 20.00 | 22.00 | 18.00 | 22.00 | 18.00 | 20.00 | 3-10 |
| 4 | Microcrystalline cellulose | 10.00 | 11.00 | 9.00 | 11.00 | 9.00 | 10.00 | 3-10 |
| 5 | Croscarmellose sodium | 10.00 | 11.00 | 9.00 | 11.00 | 9.00 | 10.00 | |

Binder

| | | | | | | | | |
|---|---|---|---|---|---|---|---|---|
| 6 | Hydroxypropyl methyl cellulose | 2.00 | 2.20 | 1.80 | 2.20 | 1.80 | 2.00 | 2-8 |
| 7 | Purified water | Qs | Qs | Qs | Qs | Qs | Qs | — |

Extra-granular

| | | | | | | | | |
|---|---|---|---|---|---|---|---|---|
| 8 | Magnesium stearate | 0.60 | 0.66 | 0.54 | 0.66 | 0.54 | 0.60 | 0.8-2 |

| | | | | | | | |
|---|---|---|---|---|---|---|---|
| Core Tablet weight | 90.00 | 98.95 | 81.10 | 102.31 | 81.30 | 90.00 | 100.00 |
| Film coating - 12% w/w suspension | 3.51 | 3.51 | 3.51 | 3.51 | 3.51 | 3.51 | — |
| Total Tablet weight | 92.70 | 102.46 | 84.61 | 105.82 | 84.81 | 92.70 | — |

*Opadry Grey (0.25 mg) contains HPMC, Titanium dioxide, Talc, ferrosoferric oxide and iron oxide yellow;
*Opadry Pink (0.5 mg) contains HPMC, Titanium dioxide, Talc, iron oxide yellow and iron oxide red;
*Opadry Tan (1 mg) contains HPMC, Titanium dioxide, Talc, ferrosoferric oxide, , iron oxide yellow and iron oxide red;
*Opadry Blue (2 mg) contains HPMC, Titanium dioxide, Talc, FD & C blue;
*Opadry Red (3 mg) contains HPMC, Titanium dioxide, Talc, FD & C red, FD & C yellow;.
*Opadry Maroon (4 mg) contains HPMC, Titanium dioxide, Talc, FD & C red, FD & C blue.

### Manufacturing Process:

1. Sifting: Co-shift brexpiprazole, corn starch, croscarmellose sodium, lactose monohydrate and microcrystalline cellulose through #40 mesh and load into RMG;
2. Binder Preparation: Add hydroxypropyl methyl cellulose into purified water under stirring;
3. Granulation: Granulate the material of step 1 with binder solution (step 2);
4. Drying: Dry the above granules until the desired LOD was achieved;
5. Milling: Mill step no 4 dried granules in suitable screen;
6. Lubrication: Lubricate the material of step 5 with Magnesium Stearate;
7. Compression: Compress the above blend with suitable punches;
8. Film coating solution preparation: Take purified water and prepare 12% w/w solution of Opadry grey (for brexpiprazole 0.25 mg), Opadry pink (for 0.5 mg), Opadry tan (for 1 mg), Opadry blue (for 2 mg), Opadry red (for 3 mg) and Opadry maroon (for 4 mg);
9. Film coating: load the compressed tablets of step 7, into coating pan and coat the tablets under coating solution of step 8, and dry the tablets;
10. Packing: pack the coated tablets by using HDPE bottles and blisters.

### Dissolution Study:

The dissolution profile of the tablets (0.2; 0.5; 1; 2; 3 & 4 mg) prepared using quantitative composition as mentioned in example 3 is shown in Table 1 below:

TABLE 1
Dissolution profile of commercially marketed tablets (REXULTI ®) and
Example 3 at particular time intervals:

| | |
|---|---|
| Time point | % drug released |

| (Minutes) | REXULTI ® | Example 3 |
|---|---|---|
| 10 | 79.8 | 85.5 |
| 15 | 88.8 | 90.0 |
| 20 | 92.0 | 92.1 |
| 30 | 94.6 | 95.4 |
| 45 | 95.7 | 97.5 |

Two dissolution profiles (REXULTI® and Example 3) are considered similar based on f1 and f2 results of above table.

### Stability Studies:

Tablet dosage form prepared in Example 3 was subjected to Accelerated stability testing as per the ICH guidelines at temperature 40°±2° C. and relative humidity of 75%±5% for 6 months. The tablet dosage form was placed in a high density polyethylene (HDPE) bottles exposed to above mentioned condition and then evaluated for impurity profile which is shown in Table 2:

TABLE 2
Results of stability tests by high performance
liquid chromatography method:

| | Example 3 | | |
|---|---|---|---|
| Impurities/Related compounds | 0 Months | 3 Months | 6 Months |
| Brexpiprazole N-Oxide | Not detected | Not detected | Not detected |
| Any individual known | Not detected | Not detected | Not detected |
| impurity | | | |
| Total impurities | Not detected | Not detected | Not detected |

The present formulation clearly indicates excellent chemical stability upon storage at accelerated stability conditions at 40°±2° C. and 75%±5% relative humidity for six months showed no evidence of any degradation products and no reduction in the content of active substance.

We claim:

1. A solid pharmaceutical composition comprising:
a) 0.1-10% w/w of brexpiprazole or its pharmaceutically acceptable salt thereof, wherein brexpiprazole has a particle size distribution such that more than 90% of the particles are between 10 μm to 40 μm;
b) 70-95% w/w of diluent selected from lactose monohydrate, corn starch and microcrystalline cellulose or combination thereof;
c) 5-15% w/w of croscarmellose sodium as disintegrant;
d) 0.5-5% w/w of hydroxypropyl methyl cellulose as binder;
e) 0.1-2% w/w of magnesium stearate as lubricant;
Wherein at least 90% of brexpiprazole dissolves within 30 minutes in a 900 ml of pH 4.3 acetate buffer at a temperature of 37±0.5° C. using a USP apparatus-2 at a paddle rotation of about 50 rpm.

2. A solid pharmaceutical composition comprising:
a) 0.1-10% w/w of brexpiprazole or its pharmaceutically acceptable salt thereof, wherein brexpiprazole has a particle size distribution such that more than 90% of the particles are between 10 μm to 40 μm;
b) 70-95% w/w of lactose monohydrate, corn starch and microcrystalline cellulose or combination thereof,
c) 5-15% w/w of croscarmellose sodium;
d) 0.5-5% w/w of hydroxypropyl methyl cellulose;
e) 0.1-2% w/w of magnesium stearate;
Wherein weight ratio of lactose monohydrate to corn starch to microcrystalline cellulose is in the range of 4:2:1 to 5:2:1 and the composition does not contains any detectable impurities by weight relative to brexpiprazole when measured by HPLC method after storage for 6 months at 40° C./75% relative humidity.

3. A solid pharmaceutical composition comprising:
a) 0.25-4 mg of brexpiprazole;
b) 40-50 mg of lactose monohydrate;
c) 20 mg of corn starch;
d) 10 mg of microcrystalline cellulose;
e) 10 mg of croscarmellose sodium;
f) 2 mg of hydroxypropyl methyl cellulose;
g) 0.6 mg of magnesium stearate;
Wherein brexpiprazole has a particle size distribution such that more than 90% of the particles are between 15 μm to 25 μm and the weight ratio of hydroxypropyl methyl cellulose to brexpiprazole is in the range of 1:0.125 to 1:2 and at least 90% of brexpiprazole dissolves within 30 minutes in a 900 ml of pH 4.3 acetate buffer at a temperature of 37±0.5° C. using a USP apparatus-2 at a paddle rotation of about 50 rpm.

4. The solid pharmaceutical composition according to claim 1, wherein the brexpiprazole is in crystalline form.

5. The solid pharmaceutical composition according to claim 1, wherein the more than 90% of the particles of brexpiprazole are between 15 μm to 25 μm, preferably 18 μm to 22 μm or 19 μm or 20 μm or 21 μm.

6. The solid pharmaceutical composition according to claim 1, wherein the loss on drying (LOD) at 105° C. is in the range of 6-10% w/w, preferably 6-8% w/w or 7.5% w/w, 7.4% w/w or 7.3% w/w or 7.2% w/w or 7.1% w/w or 7% w/w.

7. The solid pharmaceutical composition according to claim 1, wherein the weight ratio of croscarmellose sodium to brexpiprazole is 1:0.020 to 1:0.5, preferably 1:0.025 to 1:0.4.

8. The solid pharmaceutical composition according to claim 1, wherein the weight ratio of croscarmellose sodium to microcrystalline cellulose is 0.5:2.5 to 2.5:0.5, preferably 1:2, more preferably 1:1.5 or 1:1.4 or 1:1.3 or 1:1.2 or 1:1 or 1:0.9.

9. The solid pharmaceutical composition according to claim 1, wherein the weight ratio of film coating material to brexpiprazole is 1:0.01 to 1:2, preferably 1:0.05 to 1:1.5, more preferably 1:0.07 to 1:1.4.

10. The solid pharmaceutical composition according to claim 1, wherein the weight ratio of microcrystalline cellulose to brexpiprazole is 1:0.020 to 1:0.5, preferably 1:0.025 to 1:0.4.

11. The solid pharmaceutical composition according to claim 1, wherein the solid pharmaceutical composition is a tablet or capsule, if the solid pharmaceutical composition is a tablet then it is optionally coated.
